(19) United States
(12) Patent Application Publication
(10) Pub. No.: US 2017/0367982 A1
(43) Pub. Date: Dec. 28, 2017

(54) HIGH DENSITY LIPOPROTEIN FUNCTIONALIZED MAGNETIC NANOSTRUCTURES

(71) Applicant: Northwestern University, Evanston, IL (US)

(72) Inventors: Vikas Nandwana, Evanston, IL (US); Soo-Ryoon Ryoo, Evanston, IL (US); Vinayak Dravid, Evanston, IL (US); C. Shad Thaxton, Chicago, IL (US)

(21) Appl. No.: 15/632,885

(22) Filed: Jun. 26, 2017

Related U.S. Application Data
(60) Provisional application No. 62/354,438, filed on Jun. 24, 2016.

(51) Int. Cl. A61K 9/14; A61K 38/17; A61K 49/14; A61K 49/18

(52) U.S. Cl. CPC A61K 9/143; A61K 49/1824; A61K 9/145; A61K 38/1709; A61K 49/14

(57) ABSTRACT

Provided herein are compositions and methods for diagnosis and treatment of early-stage atherosclerotic plaques and reduction of plaques in arteries. In particular, provided herein are high-density-lipoprotein-functionalized magnetic nanostructures (HDL-MNS) capable of (i) precise anatomic detection of atherosclerotic lesions, (ii) removal of excess cholesterol from macrophage cells in atherosclerotic plaque, and/or (iii) delivery of therapeutic agents to plaque locations, and methods of diagnosis and treatment of atherosclerosis.

### CROSS REFERENCE TO RELATED APPLICATIONS

The present invention claims the priority benefit of U.S. Provisional Patent Application 62/354,438, filed Jun. 24, 2016, which is incorporated by reference in its entirety.

### FIELD

Provided herein are compositions and methods for diagnosis and treatment of early-stage atherosclerotic plaques and reduction of plaques in arteries. In particular, provided herein are high-density-lipoprotein-functionalized magnetic nanostructures (HDL-MNS) capable of (i) precise anatomic detection of atherosclerotic lesions, (ii) removal of excess cholesterol from macrophage cells in atherosclerotic plaque, and/or (iii) delivery of therapeutic agents to plaque locations, and methods of diagnosis and treatment of atherosclerosis.

### BACKGROUND

Heart disease is one of the leading causes of deaths in the world due to lack of early detection and targeted therapy. The main reason behind any cardiovascular event in the body is atherosclerosis, when excess fat and cholesterol in the bloodstream results in accumulation of plaque in the coronary arteries. The rupture of the vulnerable plaque can partially or completely block the flow of oxygen rich blood to heart, resulting in angina or heart attack. Targeted therapies are needed in order to control vulnerable plaque progression, and diagnosis of the atherosclerotic lesion is essential to monitor plaque size and composition before and during the therapy.

### SUMMARY

Provided herein are compositions and methods for diagnosis and treatment of early-stage atherosclerotic plaques and reduction of plaques in arteries. In particular, provided herein are high-density-lipoprotein-functionalized magnetic nanostructures (HDL-MNS) capable of (i) precise anatomic detection of atherosclerotic lesions, (ii) removal of excess cholesterol from macrophage cells in atherosclerotic plaque, and/or (iii) delivery of therapeutic agents to plaque locations, and methods of diagnosis and treatment of atherosclerosis.

In some embodiments, provided herein are high density lipoprotein magnetic nanostructure (HDL-MNS) particles, comprising: (a) a magnetic core with a hydrophobic surface; (b) a lipid layer surrounding the magnetic core; and (c) HDL-based proteins displayed on and/or embedded within the lipid layer. In some embodiments, the magnetic core comprises iron, nickel, cobalt, gadolinium, and/or manganese, and is a magnetic resonance imaging (MRI)-detectable contrast agent. In some embodiments, the hydrophobic surface comprises a saturated or unsaturated fatty acid of 4 to 24 carbon atoms. In some embodiments, the fatty acid is oleic acid. In some embodiments, the lipid layer mimics the lipid composition of natural HDLs (See, e.g, Fournier et al. Arterioscler Thromb Vasc Biol. 1997 November; 17(11):2685-91; Yetukuri et al. J Lipid Res. 2010 August; 51(8): 2341-2351; incorporated by reference in their entireties). In some embodiments, the lipid layer comprises neutral phospholipids. In some embodiments, the lipid layer 1,2-dipalmitoyl-sn-glycero-3-phosphocholine (DPPC). In some embodiments, the HDL-based proteins comprise an Apo-AI. In some embodiments, the Apo-AI comprises 70% (e.g., 70%, 75%, 80%, 85%, 90%, 95%, 100%, or ranges therebetween) sequence identity (or similarity (e.g., conservative or semi-conservative)) with wild-type human Apo-AI or a bioactive fragment thereof. In some embodiments, the HDL-MNS particles further comprise one or more additional therapeutic agents attached thereto. In some embodiments, the therapeutic agent is anchored to a lipid (e.g., sterol) inserted within the lipid layer. In some embodiments, the therapeutic agent is attached to a head group of a phospholipid that is part of the lipid layer (e.g., via direct chemical conjugation, via a linker, etc.) In some embodiments, the therapeutic agent (e.g., a hydrophobic or amphipathic agent) is loaded (e.g., without covalent attachment) into/onto the lipid layer.

In some embodiments, provided herein are high density lipoprotein magnetic nanostructure (HDL-MNS) particles, comprising: (a) a magnetic core with a hydrophilic surface; (b) HDL-based proteins coated onto the magnetic core; and (c) a lipid layer surrounding the magnetic core. In some embodiments, the magnetic core comprises iron, nickel, cobalt, gadolinium, manganese, and is a magnetic resonance imaging (MRI)-detectable contrast agent. In some embodiments, the hydrophilic surface comprises an acid component. In some embodiments, the hydrophilic surface comprises an acid component selected from the group consisting of succinic acid, glutaric acid, adipic acid, pimelic acid, suberic acid, azelaic acid, sebacic acid, dodecanedioic acid, shorter or longer linear aliphatic diacids, citric acid, isocitric acid, aconitic acid, propane-1,2,3-tricarboxylic acid, trimesic acid, itaconic acid, and maleic acid. In some embodiments, the acid is citric acid. In some embodiments, the lipid layer mimics the lipid composition of natural HDLs. In some embodiments, the lipid layer comprises neutral phospholipids. In some embodiments 2016-042 HDL_MNSs, the lipid layer 1,2-dipalmitoyl-sn-glycero-3-phosphocholine (DPPC). In some embodiments, the HDL-based proteins comprise an Apo-AI. In some embodiments, the Apo-AI comprises (e.g., 70%, 75%, 80%, 85%, 90%, 95%, 100%, or ranges therebetween) sequence identity (or similarity (e.g., conservative or semi-conservative)) with wild-type human Apo-AI or a bioactive fragment thereof. In some embodiments, HDL-MNS particles further comprise one or more additional therapeutic agent attached thereto. In some embodiments, the therapeutic agent is anchored to a lipid inserted within the lipid layer. In some embodiments, the therapeutic agent is attached to a head group of a phospholipid that comprises the lipid layer (e.g., via direct chemical conjugation, via a linker, etc.) In some embodiments, the therapeutic agent (e.g., a hydrophobic or amphipathic agent) is loaded (e.g., without covalent attachment) into/onto the lipid layer.

In some embodiments, provided herein are methods of treating or preventing atherosclerosis comprising administering to a subject an HDL-MNS particle described herein. In some embodiments, the HDL-MNS particle is administered systemically, locally to the arteries, or directly to the site of an atherosclerotic plaque. In some embodiments, the HDL-MNS particle is co-administered with another therapeutic agent or intervention for the treatment of atherosclerosis or a related disease, condition, or symptom.

In some embodiments, provided herein are methods of diagnosing, localizing, and/or characterizing atherosclerotic plaques within the arteries of a subject, comprising: (a) administering to a subject an HDL-MNS particle described herein; and (b) detecting the HDL-MNS particles within the subject by a biophysical technique. In some embodiments, the biophysical technique is magnetic resonance imaging (MRI) and the HDL-MNS particles are detected within the subject by imaging all or a portion of the subject.

In some embodiments, provided herein are methods of treating a subject for atherosclerosis and monitoring the effectiveness of the treatment, comprising: (a) administering to a subject an HDL-MNS particle described herein; (b) detecting the HDL-MNS particles within the subject by a biophysical technique at a first time-point; and (c) detecting the HDL-MNS particles within the subject by the biophysical technique at a second time-point; wherein reduction in size or number of atherosclerotic plaques between the first and second time-points indicates successful treatment. In some embodiments, methods further comprise re-administering the HDL-MNS particles prior to step (c) for detection. In some embodiments, methods further comprise therapeutically administering the HDL-MNS particles and/or another therapeutic agent between steps (b) and (c) to reduce the size or number of atherosclerotic plaques. In some embodiments, the time between the first and second time points is 1 hour, 2 hours, 3 hours, 4 hours 6 hours 12 hours, 1 day, 2 days, 3 days, 4 days, 5 days, 1 week, 2 weeks, 1 month, 2 months, 3 months, 4 months, 6 months, 9 months, 1 year, 2 years, 3 years, 4 years, any ranges therebetween.

In some embodiments, provided herein is the use of an HDL-MNS particle described herein in the treatment, prevention and/or detection of atherosclerosis.

### BRIEF DESCRIPTION OF THE DRAWINGS

FIG. 1. Synthesis of HDL-MNS-A particles (top). Oleic-acid-coated hydrophobic MNS were first coated with a neutral lipid 1,2-dipalmitoyl-sn-glycero-3-phosphocholine (DPPC), followed by coating with Apolipoprotein A1 (ApoA1). Synthesis of HDL-MNS-B particles (bottom). In a second approach, hydrophilic MNS were first coated with ApoA1 and then coated with DPPC.

FIG. 2. Circular dichroism of lipid-free Apo A-land Apo A-1 in HDL-MNS particles.

FIG. 3. r₂ relaxivity plot of HDL-MNS-B particles (left) measured at 1.4 T. Comparison of r₂ values of HDL-MNS-A and B with commercially available contrast agent; Ferumoxtran, and Ferumoxide.

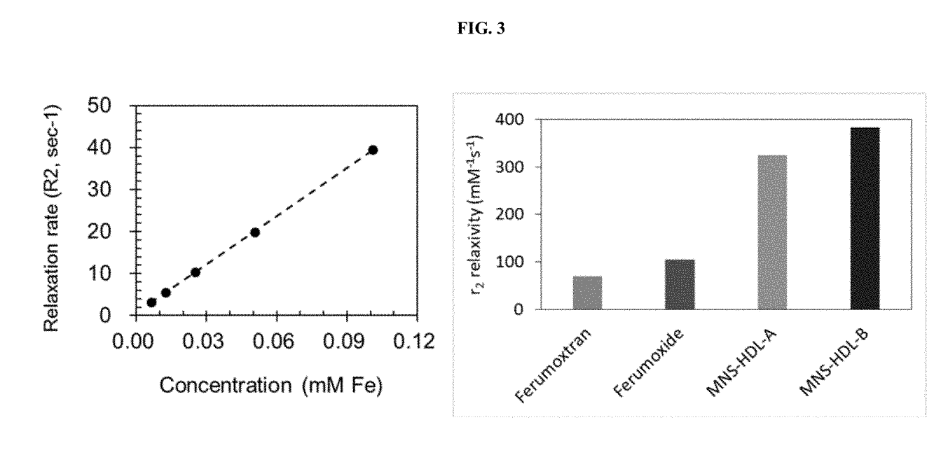

FIG. 4. Cholesterol binding isotherm curve (left) and Cholesterol efflux (right) from J774 macrophage cell lines by HDL-MNSs. HDL-MNS show high percent efflux compared to ApoA1 and serum HDL treated at similar conditions. Lipidated-MNS and citrate MNS samples did not induce efflux indicating the specificity.

FIG. 5. Cell viability of HDL-MNS A and B in J774 murine macrophage cell lines with the range of effective working concentrations (Incubation time: 24 hrs).

FIG. 6. TEM image and EDS (energy dispersive spectrum) of HDL-MNS-A uptaken by J774 macrophage cells.

FIG. 7. Magnetic resonance imaging (MRI) of cell pellets using 7T Bruker Biospin MRI. High T₂ contrast in J774 cell pellets at low concentrations of Fe used for incubation (left). A concentration dependent uptake of particles were observed from amount of Fe per cell in the samples taken from MR cell pellets determined by ICP-MS (right).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a HDL-MNS” is a reference to one or more HDL-MNS particle, unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially” refers to less than 5% variation, and preferably less than 1% variation. For example, for a structure that is “substantially spherical,” diameters in all dimensions are within 5% error of each other (and preferably within 1% of each other).

The term “about” allows for a degree of variability in a value or range. As used herein, the term “about” refers to values within 10% of the recited value or range (e.g., about 50 is the equivalent of 45-55).

As used herein, the term “theranostic” refers to the characteristic of having the combined effects of a therapeutic and a diagnostic. For example, a “theranostic agent” has utility as both a diagnostic and therapeutic agent.

As used herein, the terms “nanoparticles” and “nanostructures” are used synonymously to refer to particles having diameters in all dimensions of greater than 1 nm and less than 1 μm. Nanoparticles are often substantially-spherical, but can be of various shapes.

As used herein, the terms “magnetic nanoparticles” and “magnetic nanostructures” includes magnetic, paramagnetic, superparamagnetic, diamagnetic, ferromagnetic, and ferromagnetic materials. The nanoparticles may comprise iron, nickel, cobalt, gadolinium, manganese, and/or alloys thereof.

As used herein “diamagnetism” is the property of an object which causes it to create a magnetic field in opposition of an externally applied magnetic field causing a repulsive effect. The external magnetic field changes the magnetic dipole moment in the direction opposing the external field. Diamagnets are materials with a relative magnetic permeability less than 1. Water, wood, most organic compounds such as petroleum and some plastics, and many metals including copper, mercury, gold and bismuth are diamagnetic.

As used herein “paramagnetism” is a form of magnetism which occurs in the presence of an externally applied magnetic field. Paramagnetic materials have a relative magnetic permeability of 1 or more. Paramagnets do not retain magnetization in the absence of an externally-applied magnetic field.

As used herein “superparamagnetism” is a form of magnetism which appears in small ferromagnetic or ferromagnetic nanoparticles. The magnetic susceptibility of such materials is much larger than that of paramagnets. In the absence of external magnetic field, superparamagnetization appears to be on average zero; the superparamagnetic state. In this state, an external magnetic field is able to magnetize the nanoparticles, similarly to a paramagnet.

As used herein “ferromagnetism” is the basic mechanism by which certain materials such as iron form permanent magnets and/or exhibit strong interactions with magnets. All materials that can be magnetized by an external magnetic field and which remain magnetized after the external field is removed are either ferromagnetic or ferrimagnetic.

As used herein a “ferrimagnetic” material is one in which the magnetic moments of the atoms on different sublattices are opposed, the opposing moments are unequal and a spontaneous magnetization remains such as where different materials or ions are present in the sublattices such as Fe²⁺ and Fe³⁺. Examples of ferrimagnetic materials are YIG (yttrium iron garnet) and ferrites composed of iron oxides and other elements such as aluminum, cobalt, nickel, manganese and zinc.

As used herein, the term “lipid” refers to a variety of compounds that are characterized by their solubility in organic solvents. Such compounds include, but are not limited to, fats, waxes, steroids, sterols, glycolipids, glycosphingolipids (including gangliosides), phospholipids, terpenes, fat-soluble vitamins, prostaglandins, carotenes, and chlorophylls.

As used herein, the term “apolipoprotein” refers to a class of lipid-binding proteins which are the protein component of lipoproteins. Apoliproteins are classified in five major classes: Apo A, Apo B Apo C. Apo D, and Apo E, as known in the field.

The term “high density lipoprotein particle” (“HDL”) is used in accordance with its meaning in the field, and denotes a lipid-protein-complex with a density from about 1.06 to about 1.21 g/ml, and typically having apolipoprotein-AI as its primary protein component (although allowing for other protein components, such as apo-AII, apo-CI, apo-CII, apo-D, and apo-E).

As used herein, the term “surface exposed” refers to compounds or biomolecules that are present at the surface of a structure (e.g., nanoparticle) and are accessible to the environment surrounding the structure as well as being accessible to other agents or surfaces within the environment.

As used herein, the term “physiologic conditions” refers to solution or reaction conditions roughly simulating those most commonly found in mammalian organisms, particularly humans (e.g., not relating to specific micorenvironments within organisms (e.g., not the acidic conditions (pH 5.0) commonly found in tumor microenvironments and cellular late endosomes) or other rare conditions, unless specifically-noted). While variables such as temperature, availability of cations, and pH ranges may vary, “physiologic conditions” typically mean a temperature of 35-40° C., with about 37° C. being particularly preferred, and a pH of 7.0-8.0, with about 7.5 being particularly preferred. The conditions may also include the availability of cations, preferably divalent and/or monovalent cations, with a concentration of about 2-15 mM Mg²⁺ and 0 1.0 M Na⁺ being particularly preferred.

As used herein, the term “wild-type,” refers to a gene or gene product (e.g., protein) that has the characteristics (e.g., sequence) of that gene or gene product isolated from a naturally occurring source, and is most frequently observed in a population. In contrast, the term “mutant” refers to a gene or gene product that displays modifications in sequence when compared to the wild-type gene or gene product. It is noted that “naturally-occurring mutants” are genes or gene products that occur in nature, but have altered sequences when compared to the wild-type gene or gene product; they are not the most commonly occurring sequence. “Synthetic mutants” are genes or gene products that have altered sequences when compared to the wild-type gene or gene product and do not occur in nature. Mutant genes or gene products may be naturally occurring sequences that are present in nature, but not the most common variant of the gene or gene product, or “synthetic,” produced by human or experimental intervention.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence “having at least Y % sequence identity with SEQ ID NO:Z” may have up to X substitutions relative to SEQ ID NO:Z, and may therefore also be expressed as “having X or fewer substitutions relative to SEQ ID NO:Z.”

The term “effective dose” or “effective amount” refers to an amount of an agent which results in a desired biological outcome (e.g., inhibition of osteoclast production and/or activity).

As used herein, the terms “administration” and “administering” refer to the act of providing a therapeutic, prophylactic, or other agent to a subject for the treatment or prevention of one or more diseases or conditions. Exemplary routes of administration to the human body are through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the term “treat,” and linguistic variations thereof, encompasses therapeutic measures, while the term “prevent” and linguistic variations thereof, encompasses prophylactic measures, unless otherwise indicated (e.g., explicitly or by context).

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

The term “pharmaceutically acceptable” as used herein, refers to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

### DETAILED DESCRIPTION

Provided herein are compositions and methods for diagnosis and treatment of early-stage atherosclerotic plaques and reduction of plaques in arteries. In particular, provided herein are high-density-lipoprotein-functionalized magnetic nanostructures (HDL-MNS) capable of (i) precise anatomic detection of atherosclerotic lesions, (ii) removal of excess cholesterol from macrophage cells in atherosclerotic plaque, and/or (iii) delivery of therapeutic agents to plaque locations, and methods of diagnosis and treatment of atherosclerosis.

Naturally-occurring particles called high density lipoproteins (HDL) exhibit the capability to transfer cholesterol back from arteries to liver in a process known as reverse cholesterol transport (RCT). RCT by HDLs reduces the risk of cardiovascular disease by inhibiting the formation of atherosclerotic plaques from excess cholesterol. A recombinant HDL nanostructure with Au nanoparticle as a core has been demonstrated to be capable of binding cholesterol (Thaxton et al. JACS 2009, 131, 1384-1385; incorporated by reference in its entirety). A biodegradable synthetic HDL mimic composed of PLGA, phospholipids, and quantum dots has been used for detection of atherosclerotic plaque via optical imaging (Marrache & Dhar. Proc Natl Acad Sci USA. 2013 Jun. 4; 110(23):9445-50; incorporated by reference in its entirety). Synthetic HDL-type nanoparticles composed of Gd chelates and rhodamine based phospholipids with dual modality (magnetic and fluorescence) imaging capability have also been reported (Cormode et al. Radiology. 2010 September; 256(3):774-82; incorporated by reference in its entirety). However, each of these approaches is limited to either diagnostic or therapeutic applications. Experiments were conducted during development of embodiments herein to provide an HD-based theranostic agent for the detection and treatment of atherosclerotic plaques at an early stage.

Provided herein are theranostic agents for cardiovascular disease that are capable of precise anatomic detection as well as early treatment of vulnerable atherosclerotic lesions. In experiments conducted during development of embodiments herein, exemplary high density lipoprotein functionalized magnetic nanostructures (HDL-MNS) were synthesized by coating phospholipids and ApoA1 protein on magnetic nanostructures (MNS), mimicking outer layer of the natural HDL particles. From the diagnostic perspective, the HDL-MNS particles work as non-invasive MR imaging probes that target macrophages and allow for detection of plaques, since macrophages are key factors in the growth of atherosclerotic lesions. From the therapeutic perspective, the HDL-MNS reduce the plaque formation by removing excess amount of cholesterol from macrophage cells in and/or around atherosclerotic plaques, providing a mechanism to prevent and treat cardiovascular disease. In some embodiments, the HDL-MNS are further functionalized to carry and deliver therapeutic agents, for example, anti-inflammatory drugs, to atherosclerotic plaque to alleviate pathological symptoms.

Experiments were conducted during development of embodiments herein to generate non-invasive theranostic agents for cardiovascular disease that are capable of early-stage detection and treatment of vulnerable atherosclerotic lesions. Magnetic nanostructures functionalized with phospholipids and ApoA1 protein (HDL-MNS) have been synthesized to mimic natural HDL particles present in the body.

From the diagnostic perspective, the HDL-MNS particles show relaxivity up to 383 mM⁻¹s⁻¹, 5 times higher than commercially available MM contrast agents. Uptake of HDL-MNS particles by macrophage cells was confirmed by TEM/EDS and ICP-MS. The cells with internalized HDL-MNS were then imaged using MR scan and showed a higher T₂ contrast than commercial T₂ contrast agent. The diagnostic capability of HDL-MNS show their potential as non-invasive MR imaging probes for cardiovascular disease that can target and diagnose macrophages in atherosclerotic lesions.

From the therapeutic perspective, HDL-MNS showed higher binding affinity to cholesterol (Kd=69.9 nM) and capacity to induce cholesterol efflux (˜4.8%) from macrophage cells comparable to natural HDL (˜4.7%). The higher cholesterol efflux capacity of the HDL-MNS shows that it can reduce the plaque formation by removing excess cholesterol from macrophage cells in atherosclerotic plaques, thereby providing a mechanism to prevent and treat cardiovascular disease.

In some embodiments, provided herein are HDL-MNS particles. In some embodiments, HDL-MNS particles comprise: (A) a magnetic core, with (B) a hydrophobic exterior, surrounded by (C) a lipid layer, having (D) HDL-based protein components therein/on. In some embodiments, provided herein are HDL-MNS particles. In some embodiments, HDL-MNS particles comprise: (A) a magnetic core, with (B) a hydrophilic exterior, surrounded by (C) HDL-based protein components, and (D) a lipid layer. The HDL-MNS particles may further comprise one or more (E) therapeutic agents or (F) other components within or attached to the lipid layer. The various components of HDL-MNS particles are described below. Various embodiments within the scope herein comprise any suitable combination of the following.

In some embodiments, HDL-MNS particles comprise a magnetic core, surrounded by a hydrophobic exterior and lipid layer (e.g., which apolipoproteins therein/thereon). In some embodiments, HDL-MNS particles comprise a magnetic core, surrounded by a hydrophilic exterior and lipid layer (e.g., which apolipoproteins therein/thereon). The magnetic core is comprised of any suitable material for achieving the magnetic characteristics useful in embodiments herein. In some embodiments, the nanoparticles comprise iron, nickel, cobalt, gadolinium, manganese, etc. and/or alloys thereof. In some embodiments, the magnetic nanoparticles comprise any suitable magnetic material or combination of materials, such as magnetite, ulvospinel, hematite, ilmenite, maghemite, jacobsite, trevorite, magnesioferrite, pyrrhotite, greigite, troilite, goethite, lepidocrocite, feroxyhyte, iron, nickel, cobalt, awaruite, wairauite, barium ferrite, cobalt ferrite, nickel ferrite, manganese ferrite, strontium ferrite, zinc ferrite, or any combination thereof. Certain of the aforementioned magnetic materials are described in further detail below.

Magnetite is a ferrimagnetic mineral (Fe₃O₄), one of several iron oxides and a member of the spinel group. The common chemical name is ferrous-ferric oxide. Magnetite's chemical formula is sometimes written as FeO.Fe₂O₃, identifying it as one part wustite (FeO) and one part hematite (Fe₂O₃). Magnetite is the most magnetic of all the naturally occurring minerals on earth. Ulvospinel is an iron titanium oxide mineral (Fe₂TiO₄). It belongs to the spinel group of minerals, as does magnetite, (Fe₃O₄). Ulvospinel forms as solid solutions with magnetite at high temperatures and reducing conditions. Hematite (Fe₂O₃) is the reaction product of magnetite and oxygen. Ilmenite (crystalline iron titanium oxide, FeTiO₃) is weakly magnetic. Maghemite (Fe₂O₃, y-Fe₂O₃) is spinel in structure, the same as magnetite and is also ferrimagnetic. Its character is intermediate between magnetite and hematite. Jacobsite is a manganese iron oxide mineral, a magnetite spinel. Trevorite (NiFe³⁺₂O₄) is a rare nickeliferous mineral belonging to the spinel group. Magnesioferrite is a magnesium iron oxide mineral, a member of the magnetite series of spinels. Pyrrhotite is a iron sulfide mineral with a variable iron content: Fe_{(1.)}S (x=0 to 0.2), and is weakly magnetic. Greigite is an iron sulfide mineral with formula: Fe(II)Fe(III)₂S₄, also written as Fe₃S₄. Every molecule has one Fe²⁺ and two Fe³⁺ ions. It is a magnetic sulfide analogue of the iron oxide magnetite (Fe₃O₄). Troilite (FeS) is a variety of the iron sulfide mineral pyrrhotite. Goethite (FeO(OH) is an iron oxyhydroxide. Feroxyhyte and Lepidocrocite are polymorphs with the same chemical formula as goethite but with different crystalline structures making them distinct minerals. Awaruite (Ni₃Fe) is a nickel iron containing mineral. Wairauite (CoFe) is an iron cobalt containing mineral. Magnetic nanoparticles having the composition CoFe₂O₄ or MnFe₂O₄ or Nickel or Cobalt are also useful.

In some embodiments, the primary determinants of the choice of specific magnetic material(s) for nanoparticles depends on the ease of synthesis, the strength/type of its magnetic properties, and in some instances the ease of functionalizing its surface and/or the ease of complexing or conjugation.

In some embodiments, the magnetic nanoparticles are less than 500 nm is diameter (e.g., <400 nm, <300 nm, <250 nm, <200 nm, <150 nm, <100 nm, <65 nm, <50 nm, <40 nm, <30 nm, <25 nm, <20 nm, <18 nm, <16 nm, <15 nm, <14 nm, <13 nm, <12 nm, <11 nm, <10 nm). In some embodiments, a population of magnetic nanoparticles used in embodiments herein have a mean diameter of 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 40 nm, 50 nm, 75 nm, 100 nm, 150 nm, 200 nm, 250 nm, 300 nm, 400 nm, or any ranges therebetween (e.g., as measured by TEM). In some embodiments, a population of magnetic nanoparticles used in embodiments herein have size distribution less than 25% (e.g., <20%, <15%, <10%, <5%, <1%) (e.g., as measured by TEM).

In some embodiments, the magnetic nanoparticles used in embodiments herein are hydrophobic magnetic nanoparticles and/or magnetic nanoparticles coated with a hydrophobic layer (e.g., to allow favorable interactions between the magnetic nanoparticles and the lipid layer). In some embodiments, magnetic nanoparticles comprise a magnetic core material and an outer hydrophilic layer of hydrophobic (lipophilic) compounds on the surface. According to some embodiments, the lipophilic compound is a fatty acid. In some embodiments, a suitable fatty acid contains from 4 to 24 carbon atoms, and may be saturated or unsaturated. In some embodiments, the fatty acid is selected from palmitoleic acid, oleic acid, erucic acid, linoleic acid, linolenic acid, arachidonic acid, and ricinoleic acid.

In some embodiments, hydrophobic magnetic nanoparticles comprise a magnetic metal/alloy core and a hydrophobic exterior comprising oleic acid. Oleic acid is a preferred compound for surface-functionalization of nanoparticles (Bica D. et al. Journal of Magnetism and Magnetic Materials 2007, 311, 17-21; Lan Q. et al. Journal of Colloid and Interface Science 2007, 310, 260-269; Ingram D. R. et al. Journal of Colloid and Interface Science 2010, 351, 225-232; incorporated by reference in their entireties); although other fatty acids may be utilized in embodiments herein.

In some embodiments, the magnetic nanoparticles used in embodiments herein are hydrophilic magnetic nanoparticles and/or magnetic nanoparticles coated with a hydrophilic layer. In some embodiments, magnetic nanoparticles comprise a magnetic core material and an outer hydrophilic layer of hydrophilic (lipophobic) compounds on the surface. According to some embodiments, the hydrophilic compound is an acid. In some embodiments, a suitable acid is selected from succinic acid, glutaric acid, adipic acid, pimelic acid, suberic acid, azelaic acid, sebacic acid, dodecanedioic acid, shorter or longer linear aliphatic diacids, citric acid, isocitric acid, aconitic acid, propane-1,2,3-tricarboxylic acid, trimesic acid, itaconic acid, maleic acid, etc. In some embodiments, the magnetic nanoparticles used in embodiments herein are hydrophilic magnetic nanoparticles and/or magnetic nanoparticles coated with citric acid.

In some embodiments, the HDL-MNS particles described herein comprise a lipid layers surrounding a magnetic nanoparticle core. In various embodiments, the lipid layer may comprise suitable lipids, phospholipids, steroids (e.g., sterols), and other components useful or suitable for the formation of such a layer (e.g., a layer capable of mimicking (to at least some degree) natural HDLs), or suitable combinations thereof. For example, suitable phospholipids for inclusion in the lipid layer of HDL-MNS particles include: 1,2-Dilauroyl-sn-glycero-3-phosphocholine (DLPC), 1,2-dimyristoyl-sn-glycero-3-phosphocholine (DMPC), Dipalmitoylphosphatidylcholine (DPPC), 1,2-distearoyl-sn-glycero-3-phosphocholine (DSPC), 1,2-Dioleoyl-sn-glycero-3-phosphocholine (DOPC), 1,2-Bis(dimethylphosphino)ethane (DMPE), 1,2-Dipalmitoyl-sn-glycero-3-phosphoethanolamine (DPPE), 1,2-Dioleoyl-sn-Glycero-3-Phosphoethanolamine (DOPE), 1,2-ditetradecanoyl-sn-glycero-3-phosphate (DMPA), 1,2-Dipalmitoyl-sn-glycero-3-phosphate (DPPA), 1,2-di-(9Z-octadecenoyl)-sn-glycero-3-phosphate (DOPA), 1,2-Dimyristoyl-sn-Glycero-3-PhosphoGlycerol (DMPG), 1,2-dihexadecanoyl-sn-glycero-3-phospho-(1′-rac-glycerol) (DPPG), 1,2-dioleoyl-sn-glycero-3-phospho-(1′-rac-glycerol) (DOPG), 1,2-dimyristoyl-sn-glycero-3-phospho-L-serine (DMPS), 1,2-dipalmitoyl-sn-glycero-3-phospho-L-serine (DPPS), 1,2-dioleoyl-sn-glycero-3-phospho-L-serine (DOPS), 1,2-dioctadecanoyl-sn-glycero-3-phosphoethanolamine (DSPE), 1,2-dioleoyl-3-trimethylammonium-propane (DOTAP), etc. Similarly, suitable sterols for inclusion in the lipid layer of HDL-MNS particles include, but are not limited to: cholesterol, ergosterol, hopanoids, phytosterol, stanol, etc. Further, any of the aforementioned components may be appropriately modified (e.g., terminally modified) with moieties, e.g., for interaction with the solvent surrounding the structure or components therein. For example, one or more lipid components may be terminally modified, with a suitable moiety such as: poly(ethylene glycol) (PEG), poly(ethylene oxide)diacrylate (PEODA), polyacrylic acid, poly vinyl alcohol, collagen, poly(D, L-lactide-co-glycolide (PLGA), polyglactin, alginate, polyglycolic acid (PGA), other polyesters (e.g., poly-(L-lactic acid) (PLLA), polyanhydrides, poly(diol citrate)s, etc.), etc. Examples of polymer modified lipids include cholesterol-terminated poly(acrylic acid) (Chol-PAA) and poly(ethylene glycol) modified DSPE (e.g., PE-PEG600, PE-PEG2000, PE-PEG3000, etc.), poly(ethylene glycol) modified cholesterol (Chol-PEG), etc.

A typical HDL comprises 3-15% triglycerides (e.g., 3%, 4%, 5%, 6%, 7%, 8%, 9%, 10%, 11%, 12%, 13%, 14%, 15%, or ranges therebetween), 26-46% phospholipids (e.g., 26%, 28%, 30%, 32%, 34%, 36%, 38%, 40%, 42%, 44%, 46%, or ranges therebetween), 15-30% cholesteryl esters (e.g., 15%, 16%, 17%, 18%, 19%, 20%, 22%, 24%, 26%, 28%, 30%, or ranges therebetween), and 2-10% cholesterol (e.g., 2%, 3%, 4%, 5%, 6%, 7%, 8%, 9%, 10%, or ranges therebetween). In some embodiments, the lipid layer of the HDL-MNS particles herein mimics the lipid content ranges of natural HDLs. In other embodiment, HDL-MNS particles have lipid content ranges that are distinct from natural HDLs, but are still compatible with association with, for example, Apo-AI, and with mimicking the biological functions of HDLs (e.g., localization at atherosclerotic plaques, uptake into macrophages, removal of excess cholesterol from macrophages, etc.).

In some embodiments, the lipid layers of HDL-MNS particles described herein comprise between 70 mol % and 100 mol % phospholipid content (e.g., 70 mol %, 75 mol %, 80 mol %, 85 mol %, 90 mol %, 95 mol %, 99 mol %, 100 mol %, and ranges therebetween) within the lipid layer. In some embodiments, a single type of phospholipid is present (e.g., DPPC, DMPC, DOPC, etc.). In some embodiments, lipid-anchors (e.g., lipids (e.g., sterols) attached to agents for display on the surface of the HDL-MNS particles) comprises 1-30 mol % of the content of the lipid layer (e.g., 1 mol %, 2 mol %, 5 mol %, 10 mol %, 15 mol %, 20 mol %, 25 mol %, 30 mol %, and ranges therein (e.g., 5-20 mol %)).

In some embodiments, the lipid content of the lipid layer is 100% DPPC.

As noted herein, and understood in the field, lipoproteins are complexes of lipids (e.g., phospholipids) and lipid-binding proteins (e.g., apolipoproteins). As such, in some embodiments, the HDL-MNS particles described herein comprise apolipoproteins displayed on the surface of the lipid layer and/or bound-to/embedded-within the lipid layer.

In some embodiments, HDLs are distinguished from other lipoprotein complexes (e.g., LDLs and VLDLs) by their higher protein content (and corresponding lower lipid content) and therefore higher density. In some embodiments, HDLs comprise 50-60% protein. In some embodiments, the HDL-MNS particles herein mimic the protein content of natural HDLs. In other embodiment, HDL-MNS particles have a protein content that is distinct from natural HDLs, but is still compatible mimicking the biological functions of HDLs (e.g., localization at atherosclerotic plaques, uptake into macrophages, removal of excess cholesterol from macrophages, etc.).

Apolipoprotein A-I (apoA-I, Apo-AI, or variations thereof) is the major protein constituent of HDLs. In some embodiments, Apo-AI is the primary protein components of the HDL-MNS particles herein. In some embodiments, at least 50% (e.g., 50%, 55%, 60%, 65%, 70%, 75%, 80%, 85%, 90%, 95%, 100%, and ranges therebetween) of the protein in an HDL-MNS (e.g., on or within the lipid layer) is Apo-AI. In some embodiments, Apo-AI is the only protein component of an HDL-MNS particle.

In some embodiments, HDL-MNS particles herein comprise one or more of Apo-All, Apo-CI, Apo-CII, Apo-D, Apo-E, and/or other suitable proteins found in natural HDLs (e.g., in addition to Apo-AI). In some embodiments, any one of Apo-All, Apo-CI, Apo-CII, Apo-D, and Apo-E may comprise up to 50% e.g., 1%, 2%, 3%, 4%, 5%, 10%, 15%, 20%, 25%, 30%, 35%, 40%, 45%, 50%, or ranges therebetween) of the protein content of an HDL-MNS.

In some embodiments, HDL-MNS particles are not limited to natural protein sequences, wild-type protein sequence, or protein sequences from any particular species (e.g., human). In some embodiments, modifications to natural protein sequences (e.g., wild-type Apo-AI) may be made for any purpose. In some embodiments, a protein for use in the HDL-MNS particles herein comprise at least 60% (e.g., 60%, 65%, 70%, 75%, 80%, 85%, 90%, 95%, 100%, or ranges therebetween) sequence identity with a wild-type protein found in natural HDLs. In some embodiments, a protein for use in the HDL-MNS particles herein comprises at least 60% (e.g., 60%, 65%, 70%, 75%, 80%, 85%, 90%, 95%, 100%, or ranges therebetween) sequence similarity (e.g., conservative similarity, semi-conservative similarity) with a wild-type protein found in natural HDLs. In some embodiments, HDL-MNS particles comprise a truncated version of a natural protein (e.g., wild-type human Apo-AI). In some embodiments, the truncated portion comprises the C-terminus, N-terminus, an internal loop or non-essential domain, etc. In some embodiments, HDL-MNS particles comprise an active polypeptide or peptide fragment of a natural protein (e.g., wild-type human Apo-AI).

In some embodiments, HDL-MNS particles comprise a modified or truncated version of human Apo-AI (SEQ ID NO: 1). In some embodiments, HDL-MNS particles comprise an Apo-AI with at least 60% (e.g., 60%, 65%, 70%, 75%. 80%, 85%, 90%, 95%, 100%, or ranges therebetween) sequence identity with wild-human Apo-AI (SEQ ID NO: 1). In some embodiments, HDL-DINS particles herein comprises an Apo-AI at least 60% (e.g., 60%, 65%, 70%, 75%, 80%, 85%, 90%, 95%, 100%, or ranges therebetween) sequence similarity (e.g., conservative similarity, semi-conservative similarity) with a wild-human Apo-AI (SEQ ID NO: 1). In some embodiments, HDL-MNS particles comprise a truncated version of wild-type human Apo-AI. In some embodiments, the truncated (removed) portion comprises the C-terminus, N-terminus, an internal loop or non-essential domain, etc. In some embodiments, HDL-MNS particles comprise an active polypeptide or peptide fragment of wild-type human Apo-AI. In some embodiments, HDL-MNS particles comprise a modified and truncated version of human Apo-AI.

SEQ ID NO: 1
MKAAVLTLAVLFLTGSQARHFWQQDEPPQSPWDRVKDLATVY
VDVLKDSGRDYVSQFEGSALGKQLNLKLLDNWDSVTSTFSKL
REQLGPVTQEFWDNLEKETEGLRQEMSKDLEEVKAKVQPYLD
DFQKKWQEEMELYRQKVEPLRAELQEGARQKLHELQEKLSPL
GEEMRDRARAHVDALRTHLAPYSDELRQRLAARLEALKENGG
ARLAEYHAKATEHLSTLSEKAKPALEDLRQGLLPVLESFKVS
FLSALEEYTKKLNTQ
(1-18; signal peptide; 19-267, Preapolipo-
protein-AI; 25-267 Apolipoprotein-AI).

In some embodiments, HDL-MNS particles comprise and/or display (e.g., on or within their surface) one or more therapeutic agents (e.g., small molecules or biomolecules (e.g., peptides, polypeptides, nucleic acids, antibodies, etc.), etc.) useful for the treatment of atherosclerosis or related diseases, conditions, or disorders. The therapeutic agents may be attached to or associated with the HDL-MNS particles directly or indirectly, covalently or non-covalently, and/or by any suitable mechanism. In some embodiments, therapeutic agents are linked to anchor lipids that insert within the lipid layer of the HDL-MNS particles, thereby displaying the therapeutic agents at the surface. In some embodiments, therapeutic agents are attached to phospholipids that comprise the lipid layer. Any therapeutic agent(s) that finds use in the treatment of atherosclerosis or related diseases, conditions, or disorders may find use in embodiments herein. For example, a therapeutic agent may be a hormone, antithrombotic agent, oxidative stress inhibitor, statin, fibrinolytic agent, cholesterol lowering agents, anti-plaque agents, anti-inflammatory agent, antiproliferative agent, nitric oxide (NO), etc.

In some embodiments, a therapeutic agent is an anti-inflammatory agent. Exemplary suitable anti-inflammatory bioactive agents useful in the context of the present invention include but are not limited to steroids, such as corticosteroids like prednisone and non-steroidal anti-inflammatory drugs (or NSAIDS), such as indomethacin, aspirin and ibuprofen.

In some embodiments, HDL-MNS particles comprise additional components. For example, components for the attachment of functional moieties, for mimicry of natural HDLs, for enhancing solubility/stability/bioavailability, etc. may all be included.

In some embodiments, HDL-MNS particles comprise a cryo- and/or lyo-protecting agent. During storage of particles, the lipids, phospholipids, and other components may be susceptible to hydrolysis or other degradation. One simple way of preventing decomposition/degradation/hydrolysis is by freezing or freeze-drying. Freezing may however induce damage, for example, to the lipid layer. Addition of a cryo-protecting agent may damage from freezing or unfreezing. Examples of agents that may be used as cryo-protecting agents may without limitation be disaccharides such as sucrose, maltose and/or trehalose. Such agents may be used at various concentrations depending on the preparation and the selected agent such as to obtain an isotonic solution. In some embodiments, HDL-MNS particles are freeze-dried, stored. Dehydration generally requires use of a lyo-protecting agent such as a disaccharide (sucrose, maltose or trehalose). This hydrophilic compound prevents the rearrangement of the lipids in the formulation. Appropriate qualities for such drying protecting agents are that they possess stereo chemical features that preserve the intermolecular spacing of the lipid layer components.

In some embodiments, HDL-MNS particles comprise one or more functional surface moieties (e.g., in addition to HDL-based proteins (e.g., Apo A-I) and other components) to confer one or more beneficial functionalities to the particles. Exemplary functional moieties may include, but are not limited to: a detectable moiety (e.g., fluorophore, chromophore, contrast agent, radionuclide, etc.), a targeting/binding/interaction moiety (e.g., antibody, antibody fragment, binding peptide (e.g., recognized by a cell surface receptor), etc.), etc. For example, suitable functional moieties may include: one or more small molecules (e.g., drugs, drug-like molecules), biomolecules, a peptide or polypeptide (protein) including an antibody or a fragment thereof, a His-tag, a FLAG tag, a Strep-tag, an enzyme, a cofactor, a coenzyme, a substrate for an enzyme, a suicide substrate, a receptor, double stranded or single stranded nucleic acid (e.g., RNA or DNA), e.g., capable of binding a protein, a glycoprotein, a polysaccharide, a peptide-nucleic acid (PNA), a solid support (e.g., a sedimental particle such as a magnetic particle, a sepharose or cellulose bead, a membrane, a glass slide, cellulose, alginate, plastic or other synthetically prepared polymer (e.g., an eppendorf tube or a well of a multi-well plate, etc.), etc.), a drug (e.g., chemotherapeutic), pH sensor, a radionuclide, a contrast agent, a chelating agent, a cross-linking group (e.g., a succinimidyl ester or aldehyde, maleimide, etc.), glutathione, biotin, streptavidin, one or more dyes (e.g., a xanthene dye, a calcium sensitive dye (e.g., 1-[2-amino-5-(2,7-dichloro-6-hydroxy-3-oxy-9-xanthenyl)-phenoxy]-2-(2′-am-ino-5′-methylphenoxy)ethane-N,N,N′,N′-tetraacetic acid (Fluo-3), etc.), a sodium sensitive dye (e.g., 1,3-benzenedicarboxylic acid, 4,4′-[1,4,10,13-tetraoxa-7,16-diazacyclooctadecane-7,16-diylbis(5-methoxy-6,2-benzofurandiyl)]bis (PBFI), etc.), a NO sensitive dye (e.g., 4-amino-5-methylamino-2′,7′-difluorescein), or other fluorophore, a hapten or an immunogenic molecule (e.g., one which is bound by antibodies specific for that molecule), etc.

Functional moieties may be attached to the lipid layer, for example, via lipid anchors (e.g., lipids attached to the functional components that allow the anchor to insert in the lipid layer, leaving the functional component displayed on the particle surface). In other embodiments, functional moieties or agents may be attached to the head group of phospholipids within the lipid layer, may be attached to lipophilic moieties embedded within the bilayer (e.g., cholesterol groups), etc. Functional moieties (e.g. nitric oxide) may be directly attached to components of the lipid layer or may be connected by a suitable linker (e.g., carbon-containing chain, peptide, cleavable linker, etc.).

The HDL-MNS particles described herein may be administered to a subject per se or in the form of a pharmaceutical composition. Pharmaceutical compositions may be manufactured by means of conventional mixing, dissolving, granulating, dragee-making, levigating, emulsifying, encapsulating, entrapping or lyophilizing processes. Pharmaceutical compositions may be formulated in conventional manner using one or more physiological acceptable carriers, diluents, excipients, or auxiliaries which facilitate processing of the therapeutic compositions into preparations which can be used pharmaceutically. Proper formulation is dependent upon the route of administration chosen.

For topical administration the HDL-MNS particles may be formulated as solutions, gels, ointments, creams, suspensions etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, HDL-MNS particles may be formulated in aqueous solutions, preferably in physiologically compatible buffers such as Hank's solution, Ringer's solution, or physiological saline buffer. The solution may contain formulatory agents such as suspending, stabilizing and/or dispersing agents.

Alternatively, HDL-MNS particles may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, HDL-MNS particles may be readily formulated by combining with pharmaceutically acceptable carriers well known in the art. Such carriers enable the compounds of the invention to be formulated as tablets, pills, dragees, capsules, liquid gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium, carboxymethylcellulose, and/or polyvinylpyrrolidone (PVP); granulating agents; and binding agents. If desired, disintegrating agents may be added, such as the cross-linked polyvinylpyrrolidine, atgar, or alginic acid or a salt thereof such as sodium alginate. If desired, solid dosage forms may be sugar-coated or enteric-coated using standard techniques. For oral preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, HDL-MNS particles may take the form of tablets, lozenges, etc. formulated in conventional manner.

For administration by inhalation, the compounds for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol the dosage unit may be determined by providing a valve to deliver a metered amount.

The HDL-MNS particles are generally be used in an amount effective to achieve the intended purpose (e.g., diagnostic and/or therapeutic effect). HDL-MNS particles are administered or applied in a therapeutically effective amount. By therapeutically effective amount is meant an amount which is effective to: (1) ameliorate, or prevent the symptoms of the disease or disorder (e.g., atherosclerotic plaques), (2) allow diagnosis of the disease or condition (e.g., the presence and/or location of atherosclerotic plaques), and/or (3) prolong the survival of the patient being treated. Determination of a therapeutically effective amount is well within the capabilities of those skilled in the art, especially in light of the detailed disclosure provided herein.

For systemic administration, a therapeutically effective dose is typically estimated initially from in vitro assays. For example, a dose can be formulated in animal models to achieve a circulating concentration range that includes the IC50 as determined in cell culture; such information is used to more accurately determine useful doses in humans.

In some embodiments, HDL-MNS particles are co-administered with one or more additional therapeutic and/or diagnostic agents. In some embodiments, the co-administered agents are formulated into a single dose and/or composition. In some embodiments, the co-administered agents are in separate doses and/or compositions. In some embodiments in which separate doses and/or compositions are administered, the doses and/or compositions are administered simultaneously, consecutively, or spaced over a time span (e.g., <30 minutes, 1 hour, 2 hours, 4 hours, 6 hours, 12 hours, 1 day, 2 days, 3 days, 4 days, 5 days, 6 days, 1 week, or more, or any suitable ranges therebetween).

In certain embodiments, HDL-MNS particles are administered in an amount, expressed as a daily equivalent dose regardless of dosing frequency, of 50 micrograms (“mcg”) per day, 60 mcg per day, 70 mcg per day, 75 mcg per day, 100 mcg per day, 150 mcg per day, 200 mcg per day, or 250 mcg per day. In some embodiments, the polypeptide is administered in an amount of 500 mcg per day, 750 mcg per day, or 1 milligram (“mg”) per day. In yet further embodiments, the peptide/polypeptide/mimetic is administered in an amount, expressed as a daily equivalent dose regardless of dosing frequency, of 1-10 mg per day, including 1 mg per day, 1.5 mg per day, 1.75 mg per day, 2 mg per day, 2.5 mg per day, 3 mg per day, 3.5 mg per day, 4 mg per day, 4.5 mg per day, 5 mg per day, 5.5 mg per day, 6 mg per day, 6.5 mg per day, 7 mg per day, 7.5 mg per day, 8 mg per day, 8.5 mg per day, 9 mg per day, 9.5 mg per day, or 10 mg per day.

In various embodiments, the HDL-MNS particles are administered on a monthly dosage schedule. In other embodiments, the polypeptide is administered biweekly. In yet other embodiments, the HDL-MNS particles are administered weekly. In certain embodiments, the HDL-MNS particles are administered daily (“QD”). In select embodiments, the HDL-MNS particles are administered twice a day (“BID”).

In typical embodiments, the HDL-MNS particles are is administered for at least 1 week, at least 1 month, at least 3 months, at least 6 months, at least 12 months, or more. In some embodiments, the HDL-MNS particles are for at least 18 months, 2 years, 3 years, or more.

In some embodiments, HDL-MNS particles find use in diagnosing, assessing, treating and/or preventing the formation of atherosclerotic plaques in the arteries of a subject. In some embodiments, HDL-MNS particles find use in coronary arteries, carotid arteries, renal arteries, etc. The HDL-MNS particles may be administered systemically, locally to the arteries, and/or directly to the site (or expected or potential site) of a plaque.

The HDL-MNS particles are theranostic agents, having both therapeutic and diagnostic functionalities, and finding use in both therapeutic (e.g., treatment of atherosclerosis) and diagnostic identification/characterization/localization of atherosclerotic plaques. In some embodiments, HDL-MNS particles are employed in a combined theranostic application, if which both functionalities are utilized. In other embodiments, despite their theranostic potential, HDL-MNS particles are utilized in an application that exploits only one functionality (e.g., therapeutic or diagnostic, but not both).

In some embodiments, provided herein are methods of utilizing HDL-MNS particles for in vivo identification/characterization/localization of atherosclerotic plaques. In some embodiments, such methods comprise administering a composition comprising the HDL-MNS particles to a human or animal subject and monitoring the location of the HDL-MNS particles by a biophysical technique. In some embodiments, the biophysical technique is magnetic resonance imaging (MRI). In some embodiments, the biophysical technique is a radioimaging technique, such are positron emission tomography (PET), computed tomography (CT), or single-photon emission computed tomography (SPECT).

In some embodiments, HDL-MNS particles are used in conjunction with MRI to identify/characterize/localize atherosclerotic plaques. In some embodiments, MRI is ideally suited to provide imaging of potential atherosclerotic plaques (e.g., with HDL-MNS particles) due, at least in part, to its ability to achieve high spatial resolution without exposing the subject to ionizing radiation. Signal intensity in MR imaging is dependent on proton relaxation rates, field strength and acquisition sequence (Frullano et al. JBIC Journal of Biological Inorganic Chemistry 2007, 12, 939-949; incorporated by reference in its entirety). MR contrast agents (e.g., the HDL-MNS particles described herein) accelerate magnetic relaxation to increase contrast (Hung et al. The Journal of Physical Chemistry C 2013, 117, 16263-16273; Matosziuk et al. Inorg. Chem. 2013, 52, 12250-12261. incorporated by reference in their entireties). The association and/or preferential localization of the HDL-MNS particles at atherosclerotic plaques allows the plaques the be visualized and/or characterized (and subsequently treated) by MRI (e.g., due to the change in relaxation times).

Unlike other biophysical/diagnostic imaging techniques, such as X-ray, CT, PET, SPECT, that use ionizing radiation, MRI uses magnetic field. Since MNS are MRI contrast agents, there is no need for additional contrast agents (e.g., non-invasive). In some embodiments, MNS generate heat under RF field that finds use as a non-invasive therapy.

In some embodiments, HDL-MNS particles are administered to the expected site of potential atherosclerotic plaques. In other embodiments, HDL-MNS particles are administered systemically and allowed to localize.

In some embodiments, provided herein are methods of utilizing HDL-MNS particles for treatment of atherosclerosis and/or removal of atherosclerotic plaques. In some embodiments, such methods comprise administering a composition comprising the HDL-MNS particles to a human or animal subject and allowing the particles to mimic the effects of natural HDLs in the subject. In some embodiments, HDL-MNS particles remove fats and cholesterol from cells, within artery wall atheroma, and transport it back to the liver for excretion or re-utilization. In some embodiments, HDL-MNS particles remove cholesterol from macrophage cells at or near the site of atherosclerotic plaques. In some embodiments, HDL-MNS particles are taken-up by macrophage cells at or near the site of atherosclerotic plaques. In some embodiments, HDL-MNS particles removing cholesterol from cells (e.g., macrophage cells) at or near the site of atherosclerotic plaques, inhibiting the oxidation of low density lipoproteins (LDLs), limit inflammatory processes that underlie atherosclerosis, and/or inhibit thrombosis.

In some embodiments, HDL-MNS particles are administered for both therapeutic and diagnostic purposes. Because the HDL-MNS particles have both functionalities, they can be used to monitor the progress of treatment (e.g., treatment with HDL-MNS particles). For example, HDL-MNS particles are administered to a subject and the localization of HDL-MNS particles is monitored (e.g., imaged (e.g., by Mill)) to identify and characterize (e.g., determine the size of) atherosclerotic plaques. Then at a second time point (e.g., after hours, days, weeks, months, etc.). Localization (e.g., imaging) is repeated (with or without additional administration of HDL-MNS particles) and the atherosclerotic plaques are re-characterized. In some embodiments, HDL-MNS particles are administered as a therapeutic (e.g., without imaging) between the first and second characterization of the plaques (e.g., alone or with other therapeutics). In some embodiments, reduction in size or the plaques indicates success of the treatment.

In some embodiments, for the diagnostic, therapeutic, and/or theranostic applications described herein or understood in the field, the HDL-MNS particles may be formulated and administered according to any of the embodiments described herein.

### Experimental

### Materials and Methods

### Materials

1,2-dipalmitoyl-sn-glycero-3-phosphocholine (DPPC) and 1-palmitoyl-2-{6-[(7-nitro-2-1,3-benzoxadiazol-4-yl)amino]hexanoyl}-sn-glycero-3-phosphocholine (NBD-PC) were purchased from Avanti Polar Lipids. Apo A-I was purchased from Meridian Life Science. Alexa Fluor® 488 Protein Labeling Kit was obtained from Thermo Fisher Scientific Inc. Cell culture supplies were purchased from Invitrogen (Carlsbad, Calif.). [1, 2-³H(N)]-Cholesterol (³H-cholesterol) was obtained from Perkin-Elmer.

### HDL-MNS Synthesis

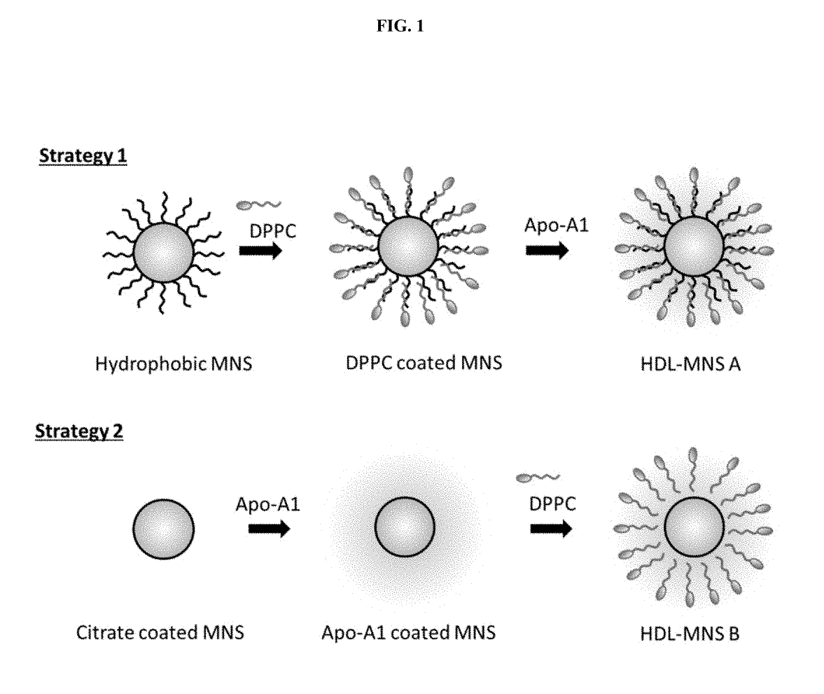

HDL-MNS particles were synthesized via two approaches. In the first approach, oleic acid coated hydrophobic MNS were dispersed in chloroform and incubated with neutral lipid 1,2-dipalmitoyl-sn-glycero-3-phosphocholine (DPPC, Avanti Polar Lipids, Inc.) dissolved in chloroform (25 mg/mL) for 30 minutes (weight ratio MNS:DPPC: 1:3). The chloroform was evaporated and water was added gradually and after sonication, DPPC coated MNS dispersed in water were obtained. Later, DPPC coated MNS were incubated with 20 fold molar excess ApoA1 and dialyzed, resulting in HDL-MNS-A particles (FIG. 1). In the second approach, the human Apo A1 (1 mg/mL, Meridian Life Sciences) was incubated with 10-fold molar excess of 8 nm of citrate coated MNS dispersed in 10 mM sodium phosphate buffer (pH 7.8) for 4˜6 hours at room temperature. Next, DPPC dissolved in ethanol (1 mg/mL) were mixed with the Apo A1-MNS solution in 20-fold molar excess and incubated for overnight with gentle agitation. To eliminate unbound protein and lipids, the solution of HDL-MNS-A and B were purified by dialysis using Pre-wetted Spectra/Por® 6 Dialysis Tubing (molecular weight cut-off, 50 kDa, Spectrum Labs, Inc.) in 10 mM phosphate buffer. The HDL-MNS concentration was measured by ICP-MS (Inductively Coupled Plasma Mass Spectrometry). Particle size distribution and zeta potentials of synthesized HDL mimic MNS were determined by Malvern Zetasizer Nano ZS90 Malvern, USA).

### APO-A1 and Phospholipid Binding

To determine the number of Apo A1 protein per HDL-MNS, fluorescence-labeled APOA1 was prepared using Alexa Fluor® 488 Protein Labeling Kit (Thermo Fisher Scientific Inc.) described in previous report (Thaxton et al., JACS, 2009, 131(4):1384-1385). The number of protein was measured based on the intensity of the labeled fluorescence signal. As compared the fluorescence signal of HDL-MNS to the standard curve obtained from the known concentration of fluorescence labeled protein. The number of loaded lipid on MNS was analyzed with similar experiments using commercially obtained fluorescently tagged lipid (1-palmitoyl-2-{6-[(7-nitro-2-1,3-benzoxadiazol-4-yl)amino]hexanoyl}-sn-glycero-3-phosphocholine, NBD-PC).

### Cell Culture

J774 cells were grown in RPMI-1640 medium containing 10% FBS and penicillin/streptomycin (100 units/mL and 100 μg/mL, respectively). The cells were cultured at 37° C. with 5% CO₂ atmosphere and plated in T75 flasks with the aforementioned media.

### Cholesterol Efflux Assay

J774 macrophage cells were used as murine cell culture model for cholesterol efflux to HDL-MNSs. The cells were seed at 15×10⁴ cells per well in 24-well plate and cultured for 24 hrs. On next day, cells were washed with PBS and incubated with 1 mCi/mL [1, 2-³H(N)]-Cholesterol for 24 hrs to label the intracellular pools of cholesterol. After removing media and washing with serum-free media, cells were exposed to HDL-MNSs for 4 hours in fresh culture media. Serum HDL, and purified ApoA1 were incubated with the cells as positive controls and lipidated MNS (L-MNS) and citrate coated MNS were used as negative controls for comparison. At the end of efflux, cell media were collected with vacuum filtration to remove floating cells and subjected to liquid scintillation counting.

### Cholesterol Binding Experiments

The cholesterol binding to HDL mimic MNS was determined by using a with a fluorescent cholesterol analogue (25-{N-[(7-nitrobenz-2-oxa-1,3-diazol-4-yl)-methyl]amino}-27-norcholesterol, NBD-cholesterol). The NBD-cholesterol solution was prepared in dimethylformamide (DMF) with varying concentrations. Fluorescence spectra of the solutions were measured after mixing 5 uL of NBD-cholesterol in DMF with 10 nM HDL-MNS in PBS and incubating for 20 min at room temperature. The solutions were excited at 473 nm and scanned from 500 to 600 nm in 1 nm increments with 1 sec integration times. The fluorescence intensity of NBD-cholesterol solution without particles were used as control and subjected to subtract the background signal. The fluorescence signal of NBD-cholesterol was detected and increased at 550 nm upon cholesterol binding, making the binding isotherm. As previously reported (Thaxton et al., JACS, 2009, 131(4):1384-1385; incorporated by reference in its entirety), equilibrium dissociation constants (K_{d}) was calculated by analyzing the binding curves with “one site total binding” function.

### Circular Dichroism Analysis

The structure of free form and conjugated Apo A1 on HDL mimic MNS under same buffer condition (10 mM sodium phosphate buffer) were analyzed by a Jasco J-815 CD spectrophotometer (JASCO). The CD spectra of nanoconstructs were also subtracted from background CD of MNS (no ApoA1) in the same buffer.

### MTS Assay for Cell Viability Test

J774 cells were plated at 2×10⁴ cells per well in 96-well plate with 70˜80% of confluency. MTS (3-(4,5-dimethylthiazol-2-yl)-5-(3-carboxymethoxyphenyl)-2-(4-sulfophenyl)-2H-tetrazolium) assay was used to quantify the cell viability according to the protocol provided by the manufacture (CellTiter 96 Aqueous One Solution Cell Proliferation Assay; Promega). Cell were incubated with HDL-MNSs at concentrations ranging from 0 to 180 μg/mL for 24 h at 37° C. Following treatment, cells were rinsed with PBS buffer briefly and further incubated with 20 μL of MTS stock solution into each well for additional 1-4 hours at 37° C. The optical densities were recorded at 490 nm and background absorbance at 700 nm was subtracted.

### Measurement of r₂ Relaxivity

R₂ relaxation time of HDL-MNS were measured at 37° C. using a Bruker mq60 NMR analyzer (1.4 T, 60 MHz) equipped with Minispec V2.51 Rev.00/NT software (Billerica, Mass.). T₂ relaxation times were measured using a simple spin echo (SE, t2_co_mb).

### MR Imaging of Cell Pellets

J774 cells were plated at 8×10⁶ cells per well in T75 flask with 70˜80% of confluency one day prior to particle treatment. Cells were cultured in various concentrations (0.1, 0.3, 1, and 3 g/ml) of MNS-HDL and Ferumoxytol (commercial available and FDA-approved iron product for MR imaging) for 24 hours. Untreated cells were used a control. Cell pellets were collected and placed in straws without bubble. After arranged, the cell pellets were imaged using 7T Bruker Biospin MM (Bruker Biospin, Billerica, Mass.).

### Results

### Synthesis of HDL-MNS

In a first approach, oleic acid coated hydrophobic MNS were coated with a neutral lipid 1,2-dipalmitoyl-sn-glycero-3-phosphocholine (DPPC) using a solvent exchange method. Later, DPPC coated MNS were incubated with ApoA1, resulting in HDL-MNS-A particles (FIG. 1, top). In the second approach, citrate coated hydrophilic MNS were incubated with ApoA1, forming ApoA1 coated MNS that was later coated with DPPC, resulted in HDL-MNS-B (FIG. 1, bottom). The particle diameters and size distribution of synthesized HDL mimic MNS were determined from TEM and DLS. The particles thus synthesized have average diameter with range of 80˜100 nm. The zeta potential of the MNS, which was ˜33.2 mV before introduction of ApoA1 and lipid, increased up to ˜14.4 mV upon addition of ApoA1 and lipid.

### Characterization of HDL-MNS

In order to determine the number of ApoA1 and phospholipid per MNS respectively, the fluorescence labeled protein and lipid were utilized.

Quantification of Apolipoprotein A1 associated with HDL-MNS was done using Alexa-fluor488 labeled protein. Alexa Fluor 488 labeling was performed according to the manufacturer's protocol (Life Technologies). The fluorescently labeled protein was purified using column chromatography. The concentration and degree of labeling was determined using absorbance measured at 280 nm and 494 nm. The protein content (molar ratio) on the particles was calculated from the intensity of fluorescence after dialyzing the HDL-MNS. As compared the fluorescence signal of HDL-MNS to the standard curve obtained from the known concentration of fluorescence labeled protein, 2.5˜3.2 ApoA1 were found to introduce per MNS on average.

The number of loaded lipid on MNS was analyzed with similar experiments using commercially obtained fluorescently tagged lipid (1-palmitoyl-2-{6-[(7-nitro-2-1,3-benzoxadiazol-4-yl)amino]hexanoyl}-sn-glycero-3-phosphocholine, NBD-PC). From the fluorescence intensity, approximately 147.7 molecules of lipid per MNS was found.

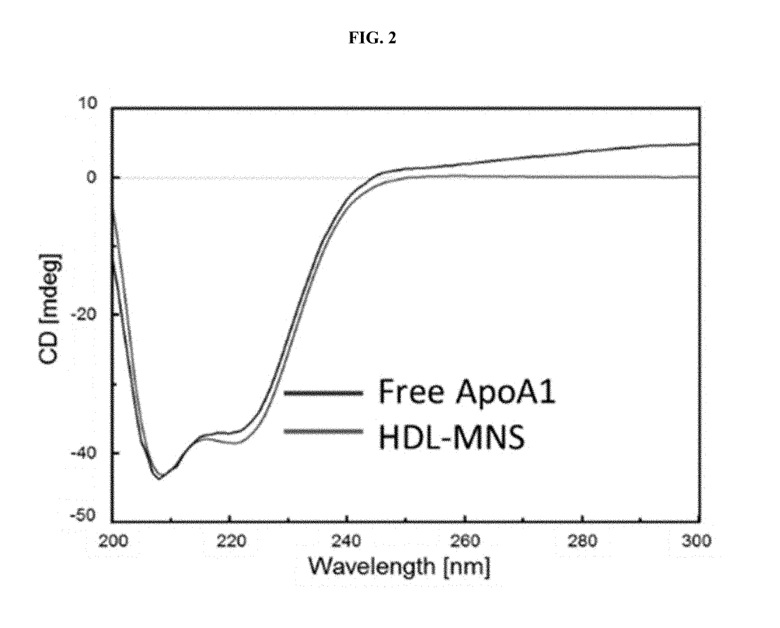

Circular dichroism was used to characterize the secondary structure of apolipoprotein to confirm its cholesterol efflux ability (FIG. 2). Lipid-free Apo A-1 was used as a control. Similar α-helicity (˜85%) of lipid-free Apo A-land Apo A-1 on MNS-HDL suggested that secondary structure of apolipoprotein in HDL-MNS was well preserved, a key criterion for the cholesterol efflux process.

The MNS used in the study have diameter size of 8 nm that shows superparamagnetic behavior. To determine r₂ relaxivity, HDL-MNS of successive dilutions in water were measured at 1.4 T with frequency 60 MHz and showed significantly high r₂ relaxivity up to 383.8 mM⁻¹s⁻¹, ˜5 times higher than r₂ of commercial available T₂ contrast agent Ferumoxtran. These high numbers indicate the MR signal generated through the HDL-MNS particles can be 5 times stronger than Ferumoxtran. Therefore, 5-times lower administration dosages of HDL-MNS particles may be used to achieve the same MR signal as Ferumoxtran.

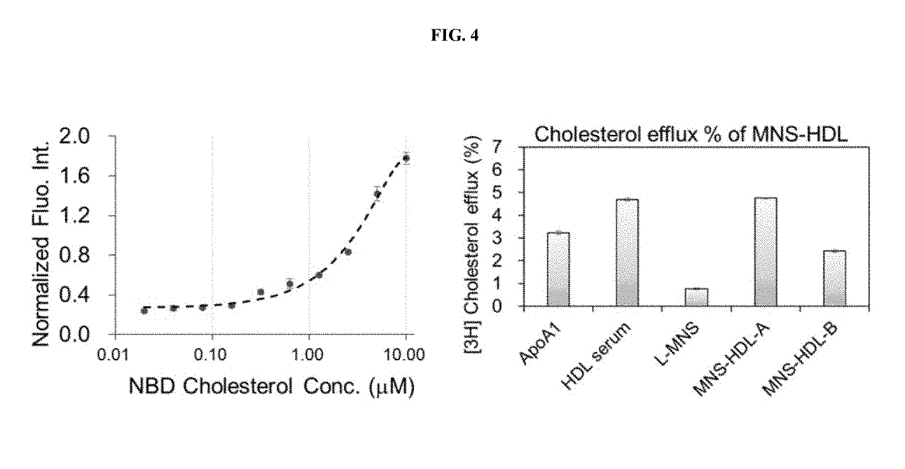

Determining the capability of HDL-MNS binding to cholesterol is important since it is required for reverse cholesterol transport. In this experiment, fluorescence intensity was used to calculate the binding of cholesterol to HDL-MNS. NBD labeled cholesterol (25-{N-[(7-nitrobenz-2-oxa-1,3-diazol-4-yl)-methyl]amino}-27-norcholesterol) was used which is a fluorescent analogue of cholesterol. It gives minimal fluorescence readout in polar environment but high fluorescence in non-polar environment. When NBD-cholesterol binds to lipid membrane of HDLs, it gives a fluorescence signal. The results suggest a strong binding of cholesterol to HDL-MNSs and increases with concentration of cholesterol and a binding isotherm was plotted from normalized fluorescence intensities. From cholesterol binding isotherm, the dissociation constant (Kd) for NBD-cholesterol binding to MNS-HDL was found to be 69.9±0.57 nM (FIG. 4, left).

The atheroprotective action of HDL can be mostly attributed to its ability to efflux cholesterol from foam cells in the atherosclerotic plaques. Experiments were conducted during development of embodiments herein to determine the ability of HDL-MNS in effluxing cholesterol from macrophages by using radiolabeled cholesterol. Two cell lines were used to determine cholesterol efflux-murine macrophages (J774) and human monocytes (THP-1). THP1 cells were differentiated to macrophages using phorbol 12-myristate 13-acetate (PMA). After labeling the cells with [³H] cholesterol, upregulation of transporters (ABCA1 and ABCG1) was induced using cAMP treatment. The cholesterol acceptors-HDL-MNS, serum HDL, or ApoA1 were incubated with the cells and radioactivity was measured in scintillation counter. Percent efflux was calculated from counts of sample versus total cell cholesterol and blank samples. From the results, it is very clear that the cholesterol efflux values from MNS-HDL were comparable with ApoA1 and natural HDL, indicates the atheroprotective of HDL-MNS (FIG. 4, right). Lipidated MNS (L-MNS) did not induce efflux indicating that ApoA1 is highly beneficial for the cholesterol efflux.

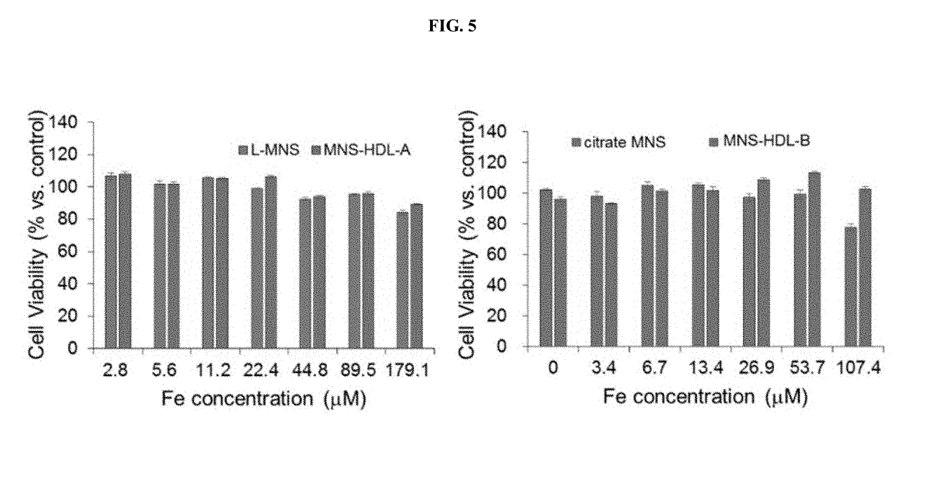

The cytotoxicity of HDL-MNS was evaluated using an MTS (3-(4,5-dimethylthiazol-2-yl)-5-(3-carboxymethoxyphenyl)-2-(4-sulfo-phenyl)-2H-tetrazolium) assay on J774 cells (FIG. 5). It is a colorimetric assay that determines the quantity of formazan end product, which is directly proportional to the number of viable cells. The ratio of absorbance in treatment wells to control was used to report percent viability of cells. From the cell viability data of macrophage cells, it is evident that HDL-MNS were nontoxic up to [Fe] 180 μM and can be used further for in vitro and in vivo studies up to these concentrations.

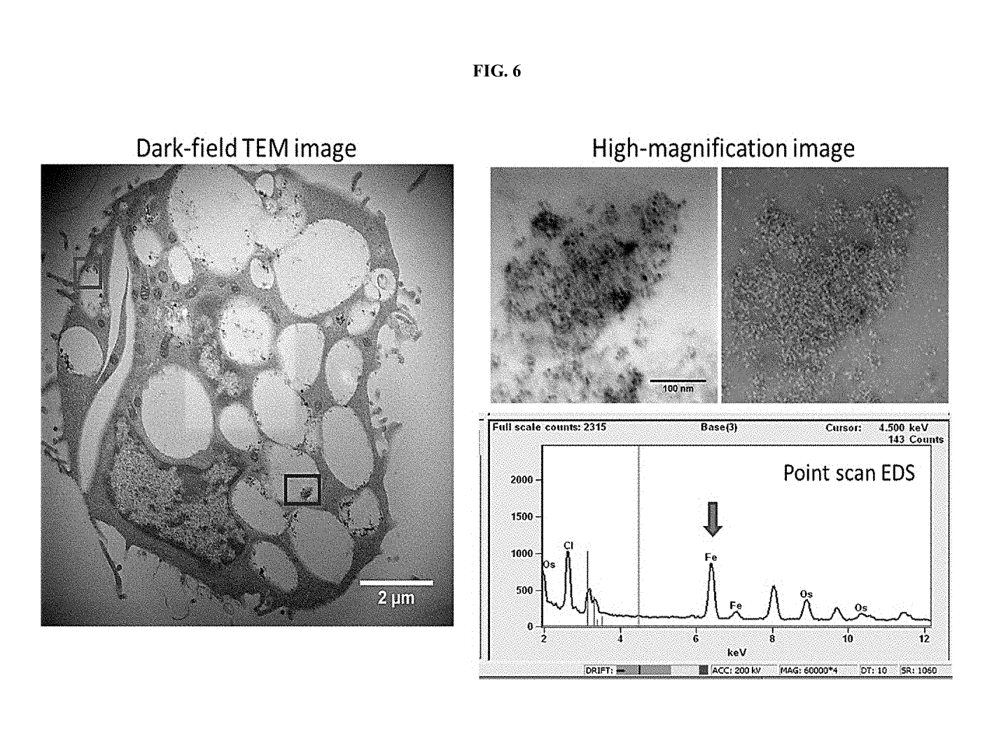

From the diagnostic perspective, HDL-MNS uptaken by the macrophage cells aids in MR imaging of atherosclerotic plaques. The cellular uptake was confirmed by TEM/EDS of the HDL-MNS particles incubated with J774 cells for 24 h (FIG. 6). Cells were fixed primarily with 2.5% glutaraldehyde and 2% formaldehyde mixture. A post-fixation was done with osmium tetroxide. After dehydrating by a series of ethanol washes, the sample is embedded in resin. Ultramicrotome sections were placed on TEM grid and images were taken. It was observed that the particles were uptaken by endocytosis and localized in vesicular structures of the cytoplasmic regions. EDS spectral and point scans show the presence of iron confirming the presence of particles inside cells.

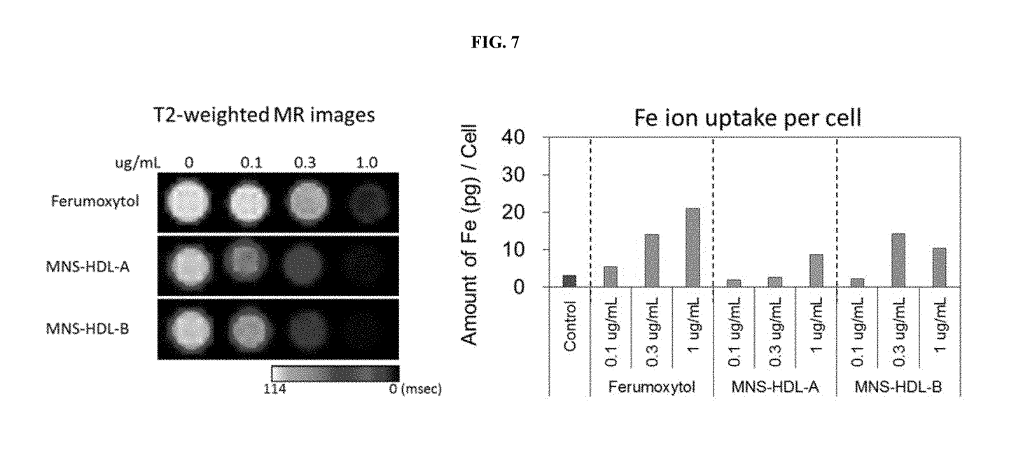

T₂-weighted MR images of HDL-MNS were taken after incubation for 24 h in J774 cells. HDL-MNS nanoparticles and Ferumoxytol (commercial FDA-approved T₂ contrast agent) were incubated in J774 cells with various concentrations (0.1, 0.3, 1, and 3 μg/mL) (FIG. 7). Cell pellets were collected and then imaged using MR scan. Untreated cells were used as a control. T₂-weighted MR phantom images show a darker signal (decrease in T₂ relaxation time) with the increase of MNS concentration. The relaxation time drop for HDL-MNS particles is significant higher than Ferumoxytol, demonstrating higher contrast enhancement properties of MNS-HDL. Fe ion uptake per cell was calculated via ICP-MS of cell pellets used for MR imaging. Concentration dependent uptake of particles was observed from amount of Fe per cell. For each sample, drop in T₂ relaxation time correlates well with increasing Fe per cell. Due to higher relaxivity, drop in T₂ relaxation time for HDL-MNS was higher than Ferumoxytol, even though the amount of Fe per cell in Ferumoxytol was higher than HDL-MNS.

All publications and patents provided herein are incorporated by reference in their entireties. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A high density lipoprotein magnetic nanostructure (HDL-MNS) particle, comprising:
(a) a magnetic core;
(b) a lipid layer surrounding the magnetic core; and
(c) HDL-based proteins displayed on and/or embedded within the lipid layer.

2. The HDL-MNS particle of claim 1, wherein the magnetic core comprises iron, nickel, cobalt, gadolinium, manganese, and is a magnetic resonance imaging (MRI)-detectable contrast agent.

3. The HDL-MNS particle of claim 1, wherein the magnetic core comprises a hydrophobic surface.

4. The HDL-MNS particle of claim 1, wherein the hydrophobic surface comprises a saturated or unsaturated fatty acid of 4 to 24 carbon atoms.

5. The HDL-MNS particle of claim 1, wherein the magnetic core comprises a hydrophilic surface

6. The HDL-MNS particle of claim 5, wherein the hydrophilic surface comprises an acid component selected from the group consisting of succinic acid, glutaric acid, adipic acid, pimelic acid, suberic acid, azelaic acid, sebacic acid, dodecanedioic acid, shorter or longer linear aliphatic diacids, citric acid, isocitric acid, aconitic acid, propane-1,2,3-tricarboxylic acid, trimesic acid, itaconic acid, and maleic acid.

7. The HDL-MNS particle of claim 1, wherein the lipid layer mimics the lipid composition of natural HDLs.

8. The HDL-MNS particle of claim 1, wherein the lipid layer comprises neutral phospholipids.

9. The HDL-MNS particle of claim 8, wherein the lipid layer 1,2-dipalmitoyl-sn-glycero-3-phosphocholine (DPPC).

10. The HDL-MNS particle of claim 1, wherein the HDL-based proteins comprise an Apo-AI.

11. The HDL-MNS particle of claim 1, further comprising a therapeutic agent anchored to a lipid inserted within the lipid layer.

12. The HDL-MNS particle of claim 1, further comprising a therapeutic agent attached to a head group of a phospholipid that comprises the lipid layer.

13. A method of treating or preventing atherosclerosis comprising administering to a subject an HDL-MNS particle of claim 1.

14. The method of claim 13, wherein the HDL-MNS particle is administered systemically, locally to the arteries system, or directly to the site of an atherosclerotic plaque.

15. The method of claim 14, wherein the HDL-MNS particle is co-administered with another therapeutic agent for the treatment of atherosclerosis or a related disease, condition, or symptom.

16. A method of diagnosing, localizing, and/or characterizing atherosclerotic plaques within the arteries of a subject, comprising:
(a) administering to a subject an HDL-MNS particle of claim 1;
(b) detecting the HDL-MNS particles within the subject by a biophysical technique.

17. The method of claim 16, wherein the biophysical technique is magnetic resonance imaging (MRI) and the HDL-MNS particles are detected within the subject by imaging all or a portion of the subject.

18. A method or treating a subject for atherosclerosis and monitoring the effectiveness of the treatment, comprising:
(a) administering to a subject an HDL-MNS particle of claim 1;
(b) detecting the HDL-MNS particles within the subject by a biophysical technique at a first time-point; and
(c) detecting the HDL-MNS particles within the subject by the biophysical technique at a second time-point;
wherein reduction in size or number of atherosclerotic plaques between the first and second time-points indicates successful treatment.

19. The method of claim 18, further comprising re-administering the HDL-MNS particles prior to step (c).

20. The method of claim 18, further comprising administering the HDL-MNS particles and/or another therapeutic agent between steps (b) and (c) to reduce the size or number of atherosclerotic plaques.


SEQUENCE LISTING (1 sequence)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ